(19) United States
(12) Patent Application Publication
(10) Pub. No.: US 2013/0250243 A1
(43) Pub. Date: Sep. 26, 2013

(54) RETINAL IMAGING DEVICE INCLUDING POSITION-SENSITIVE OPTICAL TRACKING SENSOR

(71) Applicant: VOLK OPTICAL, INC., Mentor, OH (US)

(72) Inventor: Steven D. Cech, Aurora, OH (US)

(73) Assignee: VOLK OPTICAL, INC., Mentor, OH (US)

(21) Appl. No.: 13/754,360

(22) Filed: Jan. 30, 2013

Related U.S. Application Data
(63) Continuation of application No. PCT/US2012/068079, filed on Dec. 6, 2012.
(60) Provisional application No. 61/568,323, filed on Dec. 8, 2011.

(51) Int. Cl. A61B 3/15

(52) U.S. Cl. CPC A61B 3/152
USPC 351/208

(57) ABSTRACT

A retinal imaging device is provided comprising an optical stage, one or more illumination sources, an optical system, a position-sensitive optical tracking sensor, a retinal image sensor, and a tracking controller. The illumination sources are configured to direct an illumination beam onto a cornea of an eye under examination where the illumination beam undergoes both specular and diffuse reflection. The position-sensitive optical tracking sensor comprises a non-image forming sensor configured to generate a signal indicative of the relative positioning of relatively low and high intensity portions of an optical signal incident on the sensor, in at least two dimensions. The optical system is configured to direct diffuse reflections from a cornea of an eye under examination to an input face of the position-sensitive optical tracking sensor and the tracking controller is configured to utilize an intensity distribution signal from the position-sensitive optical tracking sensor to control an optical alignment function of the optical stage, relative to a cornea of an eye under examination.

### CROSS-REFERENCE TO RELATED APPLICATIONS

The present application is filed under 35 U.S.C. 111(a) as a continuation of International Patent Application No. PCT/US12/068079, filed Dec. 6, 2012, which international application designates the United States and claims the benefit of U.S. Provisional Application Ser. No. 61/568,323 filed Dec. 8, 2011.

### BACKGROUND

The present disclosure relates to ophthalmic devices and their methods of operation including, for example, retinal imaging systems, fundus cameras, and other types of surgical and non-surgical ophthalmic devices where the human eye is under direct or indirect observation. More specifically, the present disclosure is directed towards improving the manner in which optical alignment can be achieved in such devices, making it easier to acquire clear, high-resolution images that are less subject to vignetting, shadowing, and other types of optical deficiencies.

### BRIEF SUMMARY

According to the subject matter of the present disclosure, optical systems and methods for tracking the pupil of a patient and automatically aligning the illumination and imaging optics of a retinal imaging device to the pupil are provided. Such systems and methods can be employed using relatively low cost, non image-forming optical tracking sensors and can be utilized to achieve optimum image acquisition operations.

In accordance with one embodiment of the present disclosure, a retinal imaging device is provided comprising an optical stage, one or more off-axis illumination sources, a field-limited optical system, a position-sensitive optical tracking sensor, a retinal image sensor, and a tracking controller. The off-axis illumination sources are configured to direct an illumination beam onto a cornea of an eye under examination where the illumination beam undergoes both specular and diffuse reflection. The field-limited optical system defines a detection envelope θ and primary optical axis extending from a cornea of an eye under examination through the detection envelope of the field-limited optical system. The off-axis illumination sources are displaced from the primary optical axis by a displacement angle ω that exceeds the angle of the detection envelope θ. The extent to which the displacement angle ω exceeds the angle of the detection envelope θ is sufficient to exclude a majority of specular reflections of the illumination beam from a cornea of an eye under examination and to include a significant portion of the diffuse reflections of the illumination beam from a cornea of an eye under examination. The field-limited optical system is configured to direct diffuse reflections included in the detection envelope θ to an input face of the position-sensitive optical tracking sensor and the tracking controller is configured to utilize an intensity distribution signal from the position-sensitive optical tracking sensor to control an optical alignment function of the optical stage, relative to a cornea of an eye under examination.

In another embodiment of the present disclosure, a retinal imaging device is provided comprising an optical stage, one or more illumination sources, an optical system, a position-sensitive optical tracking sensor, a retinal image sensor, and a tracking controller. The illumination sources are configured to direct an illumination beam onto a cornea of an eye under examination where the illumination beam undergoes both specular and diffuse reflection. The position-sensitive optical tracking sensor comprises a non-image forming sensor configured to generate a signal indicative of the relative positioning of relatively low and high intensity portions of an optical signal incident on the sensor, in at least two dimensions. The optical system is configured to direct diffuse reflections from a cornea of an eye under examination to an input face of the position-sensitive optical tracking sensor and the tracking controller is configured to utilize an intensity distribution signal from the position-sensitive optical tracking sensor to control an optical alignment function of the optical stage, relative to a cornea of an eye under examination.

Although the concepts of the present disclosure are described herein with primary reference to an improved retinal imaging device that includes a cost effective, optical hardware-based automatic pupil tracking and instrument alignment apparatus, it is contemplated that the concepts will enjoy applicability to any ophthalmic device where the human eye is under direct or indirect observation. For example, and not by way of limitation, it is contemplated that the concepts of the present disclosure will enjoy applicability to handheld, portable retinal imaging devices and, more generally, to retinal imaging systems, fundus cameras, auto-refractors, corneal topographers, scanning laser ophthalmoscopes, optical coherence tomographers, direct ophthalmoscopes, etc.

### BRIEF DESCRIPTION OF THE SEVERAL VIEWS OF THE DRAWINGS

The following detailed description of specific embodiments of the present disclosure can be best understood when read in conjunction with the following drawings, where like structure is indicated with like reference numerals and in which:

FIG. 1 is a schematic representation of a retinal imaging device with an optical hardware-based pupil tracking and instrument alignment apparatus as described by the present disclosure;

FIG. 2 is a schematic illustration of the iris and cornea of the eye;

FIG. 3 illustrates the specularly reflective nature of the cornea and the diffusely reflective nature of the iris;

FIG. 4 illustrates the detection envelope of the retinal imaging device of FIG. 1;

FIGS. 5 and 6 illustrate off-axis illumination sources according to embodiments of the present disclosure; and

FIGS. 7A, 7B and 7C illustrate the manner in which a linear array sensor can be utilized to generate a signal indicative of the relative positioning of relatively low and high intensity portions of an optical signal incident on the sensor, in at least two dimensions.

### DETAILED DESCRIPTION

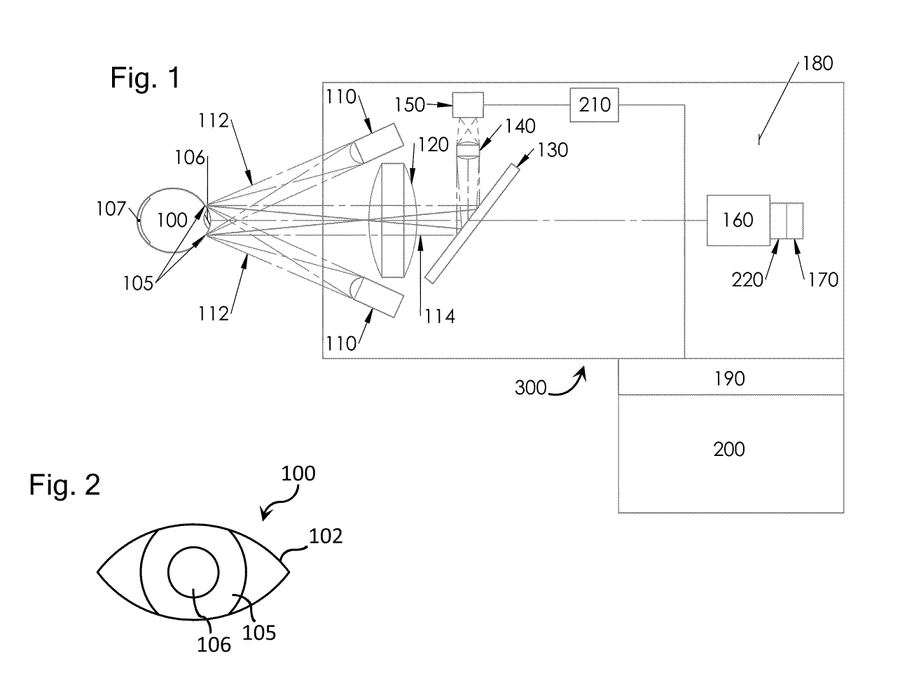

Referring initially to FIGS. 1 and 2, it is noted that a retinal imaging device 300 can be aligned coarsely with an eye 100 to be imaged via mechanical positioning of a fixed optical stage 200. The fixed optical stage 200 may be the handle or grip area of a handheld retinal camera or other imaging device. Alternatively, the fixed optical stage 200 could be the mechanical attachment point to a standard xyz joystick positioning device, as is often utilized in fixed-station fundus cameras and other conventional ophthalmic instrumentation, including retinal imaging devices or fundus cameras.

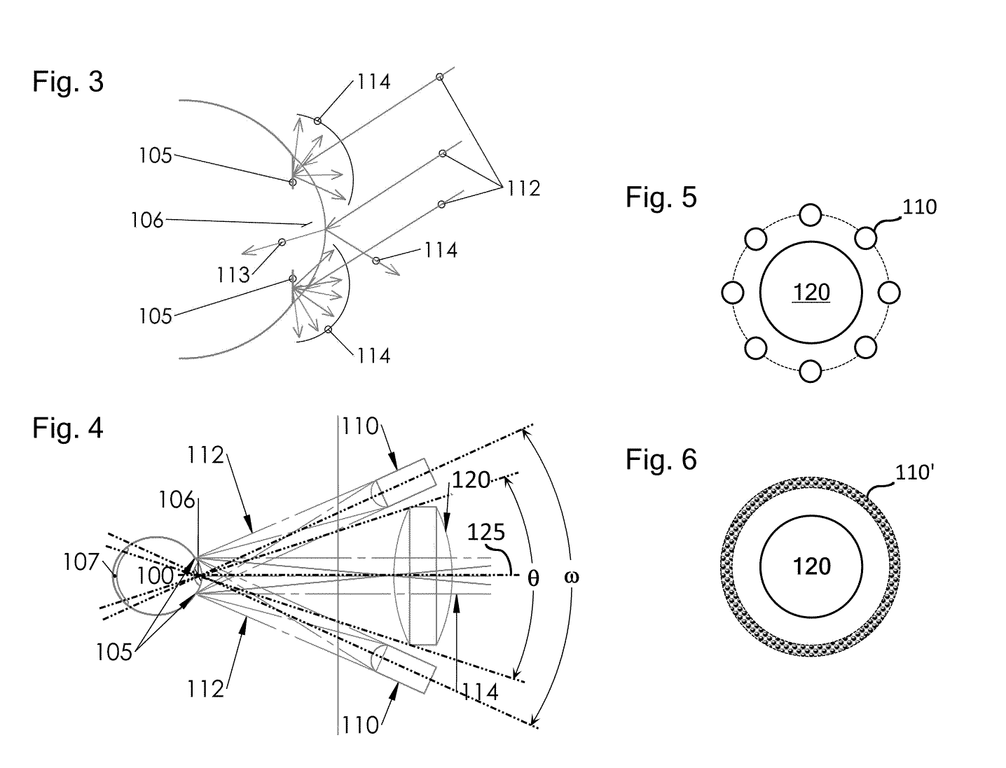

One or more off-axis illumination sources 110 can be configured optically, mechanically, and electrically to generate an intensity profile, which may be uniform or non-uniform and is directed as an illumination beam 112 onto the cornea 102 of the eye 100. At the eye 100, the illumination beam 112 selectively undergoes both specular and diffuse reflection as indicated in FIG. 3. In areas of the cornea 102 that are backed by the pupil 106 as opposed to the iris 105, individual illumination rays primarily transmit through the cornea interface, which is substantially clear relative to areas of the cornea 102 that are backed by the iris 105. A transmitted beam 113 travels through the optical media of the inner eye—the direction of transfer determined by the laws of refraction. Reflected rays form reflected illumination beams 114. The direction of travel of these individual reflected rays, which are referred to herein as specular reflections, is governed by the Law of Reflection. The magnitude of these specular reflections is a fraction of the magnitude of the original incident rays of the illumination beam 112—the exact value of which can be determined by Fresnel's Equation which governs the interaction of electromagnetic waves at the interface of dielectric materials. In areas of the cornea 102 that are backed by the iris 105, transmitted rays become incident upon the surface of the iris 105 where diffuse reflectance occurs. In diffuse reflectance, incident rays possessing unique directions of travel are reflected into a broad range or distribution of directions of travel and also form a reflected illumination beam 114. These reflected rays are referred to herein as diffuse reflections. These two types of reflections, namely, specular and diffuse reflections, are illustrated schematically in FIG. 3.

According to particular embodiments of the present disclosure, the primary ophthalmic lens 120, the beamsplitter 130, and the focusing lens 140 collectively define a field-limited optical system that is configured to exclude a majority of the specular reflections of the illumination beam 112, i.e., those portions of the reflected illumination beam 114 that originate solely from areas of the cornea 102 that are not backed by the iris 105 or another diffuse reflecting background material, and to include a substantial portion of the diffuse reflections of the illumination beam 112, i.e., those portions of the reflected illumination beam 114 that originate from areas of the cornea 102 that are backed by the iris 105 or another diffuse reflecting background material. As such, the field-limited optical system of FIG. 1 can be designed such that the illumination beam 112 and the reflected illumination beam 114, as defined and constrained by the off-axis illumination sources 110, the primary ophthalmic lens 120, the beamsplitter 130, and the focusing lens 140, allow the formation of a very unambiguous optical representation of the iris 105 and pupil 106 of the eye 100. More specifically, a relatively small portion of reflected rays originating from the area of the cornea 102 that is backed by the pupil 106 will be reflected back in directions that fall within the detection envelope of the field-limited optical system. Accordingly, the optical intensity corresponding to the pupil 106, when processed by the focusing lens 140 with support from the primary lens 120 and beamsplitter 130, will have a relatively low intensity (relatively dark). By contrast, in areas of the cornea 102 that are backed by the iris 105, diffuse reflection ensures that a relatively large portion of reflected rays originating from the iris 105 will fall within the detection envelope of the field-limited optical system. Therefore, the optical intensity corresponding to the iris 105 will have a relatively high intensity (relatively bright).

The present inventors have further recognized that, in the near-IR region of the electromagnetic spectrum, e.g., between 700 nm and 1100 nm, the wavelength-specific absorption behavior of typical iris pigments is minimized. Accordingly, in the near-IR, the reflectivity of the respective irises of most patients will be very similar and it is contemplated that near-IR sources will be particularly well-suited for use with the field limited optical system described above to make the generally circular iris 105 a very robust target to track using a relatively simple position-sensitive optical tracking sensor 150.

In the embodiment as indicated in FIG. 1, the off-axis illumination sources 110 are configured as two or more discrete elements located peripheral to the primary lens 120. Any number of off-axis illumination sources 110, including a single device, can be envisioned to be suitable in creating the illumination beam 112. Although in the illustrated embodiments, the off-axis illumination sources 110 appear as a pair of off-axis sources 110 (see FIG. 1) or a circular array of off-axis sources 110 (see FIG. 5), one advantageous implementation takes the form of a substantially continuous ring of light 110′ that extends unbroken around the complete periphery of the central primary lens 120 (see FIG. 6).

Referring to FIG. 4, more important than the number of off-axis illumination sources 110 deployed is their geometric placement relative to the other optical elements such as the primary lens 120, beamsplitter 130, and focusing lens 140. As is noted above, to create the advantageous discrimination between the pupil 106 and the iris 105, it is often advantageous to position the off-axis illumination sources 110 in positions which place any and all specular reflections occurring at the surface of the cornea outside the detection envelope θ of the field-limited optical system. This detection envelope θ is illustrated schematically in FIG. 4 as corresponding to the input acceptance angle of the lens 120 relative to the centroid of the corneal surface of the eye 100. In FIG. 4, the off-axis illumination sources 110 are displaced from the primary optical axis 125 by a displacement angle ω that exceeds the angle of the detection envelope θ. In this way, the binary dark/light intensity representation of the circular pupil 106 against the iris 105 is maintained within the reflected illumination beam 114.

In practice, care should be taken to ensure that the displacement angle ω exceeds the angle of the detection envelope θ by an amount that is sufficient to keep a majority of the specular reflections from the surface of the cornea 102 from falling within the detection envelope θ and achieve sufficient contrast in the dark/light intensity representation within the reflected illumination beam 114. Conversely, the degree to which the displacement angle ω exceeds the angle of the detection envelope θ cannot be so large as to exclude a significant portion of the diffuse reflections of the illumination beam 112, i.e., those originating from areas of the cornea 102 that are backed by the iris 105 or another diffuse reflecting background material, from the detection envelope θ. Although the detection envelope θ is illustrated in FIG. 4 as being defined by the primary lens 120, it could alternatively be defined by one or more other optical constraints in the field-limited optical system of the present disclosure. For example, and not by way of limitation, the detection envelope θ could be defined by the primary ophthalmic lens 120, the beamsplitter 130, the focusing lens 140, the position-sensitive optical tracking sensor 150, or combinations thereof.

In one embodiment of the retinal imaging device 300, near-IR LEDs are used to implement the off-axis illumination sources 110. There are commercially-available near-IR LEDs available that emit at several different wavelengths. These near-IR LEDs are offered in a variety of different types of both standard and custom optomechanical packages. Near-IR LEDs are robust and are generally easy to spatially-deploy. Additionally, they operate using low voltage DC power. Although LEDs are described as an optimum choice, other off-axis illumination sources 110 could also be used within the retinal imaging device 300 within the spirit of this disclosure. These alternate illumination sources include visible light LEDs, lamps such as halogen, metal halide, and xenon, as well as fiber optic-coupled lamps or LED sources.

As the reflected transmission beam 114 moves away from the eye 100 and in the direction of the retinal imaging device 300, it first encounters the primary lens 120. In the illustrated embodiment, the pupil tracking apparatus is implemented co-linear with the retinal imaging optics. In FIG. 1, the retinal imaging optics are schematically indicated by the presence of the primary lens 120, a retinal imaging lens 160, a focus coupler 220, and an image sensor 170. The operation of retina illumination and imaging optics within fundus cameras is well known in the art. As such, details related to the retinal image forming portion of the retinal imaging device 300 are, for the most part, omitted from this discussion.

The primary lens 120 can be optimized to generate an indirect image of the retina surface 107 somewhere between the primary lens 120 and the retina imaging lens 160. This indirect image is then relayed onto the image sensor 170 by the retina imaging lens 160. In the embodiment shown in FIG. 1, a beamsplitter 130 is used to re-direct the reflected illumination beam 114 away from the main optical pathway used for retinal imaging (retina illumination and imaging beam pathways omitted for clarity). Beamsplitters 130 as indicated in FIG. 1 are well known in the art. These devices are typically designed to allow a portion of the incident irradiation to pass through while reflecting, minor-like, the remainder of the electromagnetic radiation. Of particular applicability to the present disclosure are beamsplitters of the type that selectively transmit or reflect irradiation based on wavelength. These types of beamsplitters, known as dichroic beamsplitters, can be configured to transmit at high-efficiencies light irradiation up to a design transition wavelength while reflecting longer wavelength irradiation. It is contemplated that operation of the basic retina imaging function is advantageously performed with visible and near-IR illumination out to about 850 nm. Efficient LEDs exist that emit electromagnetic radiation out to 940 nm and beyond. In one contemplated embodiment, the off-axis illumination sources 110 are 940 nm LEDs and the beamsplitter 130 is designed to transition from transmission to reflection somewhere around 900 nm. Implemented in this way, the functions of pupil tracking and retina imaging are cleanly split from the retinal rays at the beamsplitter 130.

After reflecting off of beamsplitter 130, the reflected illumination beam 114 is brought to a focus by the focusing lens 140. Focusing lens 140 works in combination with the optical power applied to the illumination beam 114 by the primary lens 120 to bring a relatively high-contrast intensity distribution representing areas of the eye corresponding to the pupil 106 and the iris 105 into focus onto the active surface of the position-sensitive optical tracking sensor 150. Suitable tracking sensors 150 include, but are not limited to, linear array sensors such as the S5668 series 16-element Si photodiode linear array available from Hamamatsu Photonics K.K., quadrant sensors such as a low dark current quadrant photodiode available from Pacific Silicon Sensor, Inc, or any other type of position-sensitive optical sensor that can be used to generate a signal that indicates the relative positioning of relatively low and high intensity portions of an optical signal incident on the sensor, in at least two dimensions.

Regardless of the type of position-sensitive optical tracking sensor 150 is used, the electrical signals that are generated by the position-sensitive optical tracking sensor 150 can be communicated to a tracking controller 210, which is in communication with an alignment actuator 190 coupled to the optical stage 200. The tracking controller 210 can consist of, in part or in whole, analog amplifiers suitably configured to provide the appropriate sum, difference, comparison, and other signals indicative of the intensity profile at the tracking sensor 150. Additionally, the controller 210 could include a variety of other simple electronic components including mixed-signal and discrete electrical components, programmable logic devices, microcontrollers, microprocessors, power amplifiers, and motor control circuits. All of these components and their application in actuator control circuits and assemblies are well documented in the art. The output of the controller 210 comprises electrical signals that are suited to drive the specific type of actuators contained within the alignment actuator 190.

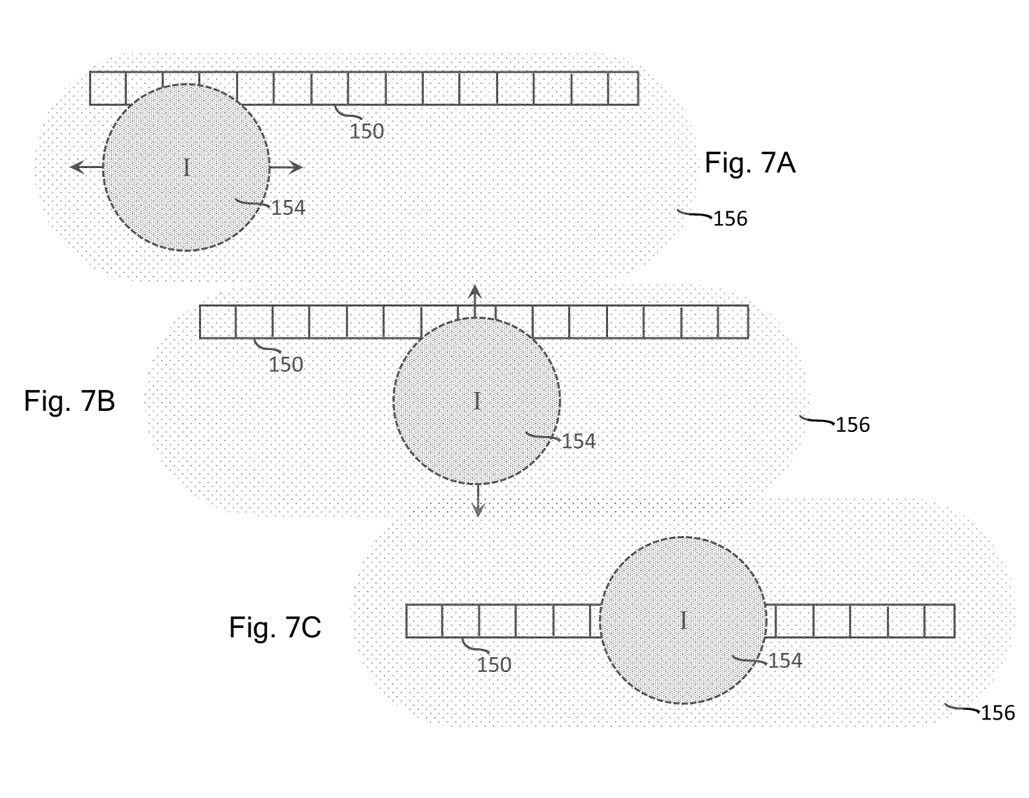

According to embodiments of the present disclosure that utilize a tracking sensor that produces a one-dimensional intensity profile, as is the case with the linear array sensor 150 illustrated in FIGS. 7A-7C, it is contemplated that a pupil tracking optical system can be configured to generate a signal that indicates the position on an input face of the sensor 150 of relatively low and high intensity portions of an optical signal incident on the sensor 150, in two dimensions. More specifically, the linear array sensor array 150 comprises a linear array of sensor elements 152 and the off-axis positioning of the illumination sources creates a beam spot characterized by a unique one-dimensional intensity profile I. This intensity profile I is derived from the off-axis configuration of the illumination sources 110 and from the optical characteristics of the cornea 102 and underlying iris 105 and includes a relatively low intensity portion 154 that is surrounded, or at least bounded on one or more sides, by a relatively high intensity portion 156.

As is illustrated schematically in FIGS. 7A-7C, the tracking controller 210 can be programmed to implement a relatively simple processing scheme to provide an indication of the centerpoint of the intensity profile I and control an alignment actuator 190 of the optical stage 200 to effect movement of the profile centerpoint along the linear array 150 of sensor elements 152 until the profile centerpoint reaches an “aligned” position. This transition to a first aligned position is illustrated as the intensity profile I moves from an unaligned position in FIG. 7A to the aligned position of FIG. 7B, where the system optics are aligned, in one dimension, with the pupil 106 under examination. Once the beam spot is tracked to a target location on the sensor array 150 in one dimension, i.e., a location corresponding to the center of the pupil 106, tracking control can be shifted to adjust the position of the beam spot in a second dimension, i.e., transversely across the linear array 150 of sensor elements 152. This adjustment will typically be along an axis that is perpendicular to the linear axis of the sensor array 150 and is illustrated schematically in FIG. 7C. Again, the tracking controller 210 can be programmed to implement a relatively simple processing scheme to provide an indication of the transverse centerpoint of the intensity profile I and control the alignment actuator 190 of the optical stage 200 to effect movement of the transverse centerpoint across the linear array 150 of sensor elements 152 until the profile centerpoint reaches an “aligned” position, where the system optics are aligned, in a second dimension, with the pupil 106 under examination. Once the beam spot is tracked to a target location along this additional axis, i.e., a location corresponding to the center of the pupil along a second dimension, dual axis alignment of the optical system is achieved.

According to embodiments of the present disclosure that utilize a tracking sensor that produces a two-dimensional intensity profile, as is the case with a quadrant array sensor comprising at least four sensor elements arranged symmetrically about a common sensor centroid, it is contemplated that the tracking controller 210 can be programmed to utilize signals indicative of the symmetry of the intensity profile across the sensor elements to control the alignment actuator 190 of the optical stage 200 to affect movement of a profile centroid towards the sensor centroid and align the optical system with the pupil under examination. For example, a relatively simple processing scheme of summing the signal coming from the individual detector quadrants while at the same time calculating the difference in the signal generated from two opposed detector elements can be a very robust method of generating appropriate 2-axis alignment control signals.

Generally, the alignment actuator 190 would be configured to move in at least two spatial dimensions as referenced to the fixed optical stage 200, either independently or simultaneously. The alignment actuator 190 is used to respond to pupil tracking information provided by the position-sensitive discrete optical sensor arrangement 150 and controller 210 by physically aligning the optical tube 180 of the retinal imaging system 300 with the pupil 106 and iris 105 of the eye 100. By doing this automatically, the critical fine alignment of the device is no longer limited by the positioning skills of the operator. By providing automatic closed-loop alignment at response times shorter than typical human eye or hand jitter response times, the technology of the present disclosure facilitates proper operation of the retinal imaging device 300 allowing improved image quality due to improvements in lighting uniformity and image focus actuation.

There are many different methods of supplying a suitable alignment actuator 190 that are known in the art. The alignment actuator 190 can generally be configured to provide motion in two or more independent axes. The Cartesian coordinates x and y defined to form a plane that generally is parallel to the iris 105 is one useful manner in which to configure the alignment actuator 190. Additionally, a third axis, z, of automated motion defined to be generally parallel to the reflected illumination beam 114 is advantageous in providing additional alignment fidelity. Alternately, the alignment actuator 190 could equally be configured to provide tilt and pitch actuation, or in 3 dimensions, tilt, pitch and roll actuation of the optical tube 180 relative to fixed optical stage 200.

Referring to the elements of FIGS. 1, 2, and 3, a contemplated method of providing an automated pupil tracking and instrument alignment function in support of the general operation of an improved retinal imaging device includes the following steps, which may be taken in succession:
(1) Coarse position the retinal imaging device 300 relative to the eye 100;
(2) Illuminate the complete area of the pupil 106 and iris 105 with one or more off-axis illumination sources 110;
(3) Receive the reflected illumination beam 114;
(4) Focus the reflected illumination beam 114 onto the active surface of a position-sensitive optical tracking sensor 150 via the focusing lens 140;
(5) Communicate the output of the position-sensitive optical tracking sensor 150 to a processing unit 210;
(6) Calculate the motion control drive signals required to keep retinal imager 300 properly aligned on the centroid of the pupil 106 or iris of the eye 100;
(7) Communicate motion control drive signals to the alignment actuator 190; and
(8) Automatically enact the necessary motion to align the optical tube 180 and retinal imaging device 300 to the eye 100.
   The aforementioned steps may be taken in succession or may be condensed or expanded without departing from the scope of the present disclosure.

It is noted that recitations herein of “at least one” component, element, etc., should not be used to create an inference that the alternative use of the articles “a” or “an” should be limited to a single component, element, etc.

It is noted that recitations herein of a component of the present disclosure being “configured” in a particular way, to embody a particular property, or to function in a particular manner, are structural recitations, as opposed to recitations of intended use. More specifically, the references herein to the manner in which a component is “configured” denotes an existing physical condition of the component and, as such, is to be taken as a definite recitation of the structural characteristics of the component.

It is noted that terms like “preferably,” “commonly,” and “typically,” when utilized herein, are not utilized to limit the scope of the claimed invention or to imply that certain features are critical, essential, or even important to the structure or function of the claimed invention. Rather, these terms are merely intended to identify particular aspects of an embodiment of the present disclosure or to emphasize alternative or additional features that may or may not be utilized in a particular embodiment of the present disclosure.

For the purposes of describing and defining the present invention it is noted that the terms “substantially” and “approximately” are utilized herein to represent the inherent degree of uncertainty that may be attributed to any quantitative comparison, value, measurement, or other representation. The terms “substantially” and “approximately” are also utilized herein to represent the degree by which a quantitative representation may vary from a stated reference without resulting in a change in the basic function of the subject matter at issue.

Having described the subject matter of the present disclosure in detail and by reference to specific embodiments thereof, it is noted that the various details disclosed herein should not be taken to imply that these details relate to elements that are essential components of the various embodiments described herein, even in cases where a particular element is illustrated in each of the drawings that accompany the present description. Rather, the claims appended hereto should be taken as the sole representation of the breadth of the present disclosure and the corresponding scope of the various embodiments described herein. Further, it will be apparent that modifications and variations are possible without departing from the scope of the invention defined in the appended claims. More specifically, although some aspects of the present disclosure are identified herein as preferred or particularly advantageous, it is contemplated that the present disclosure is not necessarily limited to these aspects.

It is noted that one or more of the following claims utilize the term “wherein” as a transitional phrase. For the purposes of defining the present invention, it is noted that this term is introduced in the claims as an open-ended transitional phrase that is used to introduce a recitation of a series of characteristics of the structure and should be interpreted in like manner as the more commonly used open-ended preamble term “comprising.”

1. A retinal imaging device comprising an optical stage, one or more off-axis illumination sources, a field-limited optical system, a position-sensitive optical tracking sensor, a retinal image sensor, and a tracking controller, wherein:
the off-axis illumination sources are configured to direct an illumination beam onto a cornea of an eye under examination where the illumination beam undergoes both specular and diffuse reflection;
the field-limited optical system defines a detection envelope θ and primary optical axis extending from a cornea of an eye under examination through the detection envelope of the field-limited optical system;
the off-axis illumination sources are displaced from the primary optical axis by a displacement angle ω that exceeds the angle of the detection envelope θ;
the extent to which the displacement angle ω exceeds the angle of the detection envelope θ is sufficient to exclude a majority of specular reflections of the illumination beam from a cornea of an eye under examination and to include a significant portion of the diffuse reflections of the illumination beam from a cornea of an eye under examination;
the field-limited optical system is configured to direct diffuse reflections included in the detection envelope θ to an input face of the position-sensitive optical tracking sensor; and
the tracking controller is configured to utilize an intensity distribution signal from the position-sensitive optical tracking sensor to control an optical alignment function of the optical stage, relative to a cornea of an eye under examination.

2. A retinal imaging device as claimed in claim 1 wherein the position-sensitive optical tracking sensor comprises a non-image forming sensor that is configured to generate a signal indicative of the position on an input face of the sensor of relatively low and high intensity portions of an optical signal incident on the sensor, in at least two dimensions.

3. A retinal imaging device as claimed in claim 1 wherein the position-sensitive optical tracking sensor comprises a linear array sensor that is configured to generate a one-dimensional intensity profile.

4. A retinal imaging device as claimed in claim 3 wherein:
the linear array sensor comprises a linear array of sensor elements;
the tracking controller is programmed to utilize signals indicative of a centerpoint of the one-dimensional intensity profile to control an alignment actuator of the optical stage for movement in a direction corresponding to movement of the profile centerpoint along the linear array of sensor elements; and
the tracking controller is further programmed to utilize signals indicative of a transverse centerpoint of the one-dimensional intensity profile to control the alignment actuator of the optical stage for movement in a direction corresponding to movement of the transverse centerpoint transversely across the linear array of sensor elements.

5. A retinal imaging device as claimed in claim 4 wherein the tracking controller is programmed to control the alignment actuator for independent or simultaneous movement of the profile centerpoint along the linear array of sensor elements and the transverse centerpoint across the linear array of sensor elements.

6. A retinal imaging device as claimed in claim 1 wherein the position-sensitive optical tracking sensor comprises a quadrant array sensor configured to provide a two-dimensional intensity profile.

7. A retinal imaging device as claimed in claim 6 wherein:
the quadrant array sensor comprises at least four sensor elements arranged symmetrically about a common sensor centroid; and
the tracking controller is programmed to utilize signals indicative of intensity profile symmetry across the sensor elements to control an alignment actuator of the optical stage for movement in directions corresponding to movement of a profile centroid towards the sensor centroid.

8. A retinal imaging device as claimed in claim 1 wherein the detection envelope θ is defined by an input acceptance angle of a primary ophthalmic lens of the field-limited optical system, a beamsplitter of the field-limited optical system, a focusing lens of the field-limited optical system, the position-sensitive optical tracking sensor, or combinations thereof.

9. A retinal imaging device as claimed in claim 1 wherein the field-limited optical system comprises a primary ophthalmic lens, a focusing lens, and a beamsplitter that is configured to direct diffuse reflections from iris-backed areas of the cornea of the eye under examination to a focusing lens that is optically coupled to the position sensitive optical tracking sensor.

10. A retinal imaging device as claimed in claim 9 wherein:
the off-axis illumination sources comprise near-IR illumination sources; and
the beamsplitter comprises a wavelength sensitive beamsplitter that is configured to selectively direct near-IR wavelengths to the focusing lens and the position sensitive optical tracking sensor.

11. A retinal imaging device as claimed in claim 9 wherein the off-axis illumination sources are configured as two or more discrete elements located peripheral to the primary ophthalmic lens.

12. A retinal imaging device as claimed in claim 9 wherein the off-axis illumination sources are configured as a circular array of discrete elements extending about a periphery of the primary ophthalmic lens.

13. A retinal imaging device as claimed in claim 9 wherein the off-axis illumination source is configured as a substantially continuous ring of light that extends about a complete periphery of the primary ophthalmic lens.

14. A retinal imaging device as claimed in claim 1 wherein the optical stage is the handle or grip area of a handheld retinal camera or other handheld imaging device.

15. A retinal imaging device as claimed in claim 1 wherein the optical stage is a mechanical attachment point to an xyz positioning device of a fixed-station fundus camera or a fixed-station retinal imaging device.

16. A retinal imaging device as claimed in claim 1 wherein the optical stage comprises an alignment actuator that is configured to provide motion along two or more independent axes.

17. A retinal imaging device as claimed in claim 16 wherein the optical stage comprises an alignment actuator that is configured to provide tilt and pitch actuation.

18. A retinal imaging device as claimed in claim 1 wherein the optical stage comprises an alignment actuator that is configured to provide motion along at least three independent axes x, y, z, one of which is generally parallel to the primary optical axis of the field-limited optical system.

19. A retinal imaging device as claimed in claim 1 wherein the tracking controller and the optical stage are configured to provide automatic closed-loop alignment at response times that are substantially shorter than human eye or hand jitter response times.

20. A retinal imaging device comprising an optical stage, one or more illumination sources, an optical system, a position-sensitive optical tracking sensor, a retinal image sensor, and a tracking controller, wherein:
the illumination sources are configured to direct an illumination beam onto a cornea of an eye under examination where the illumination beam undergoes both specular and diffuse reflection;
the position-sensitive optical tracking sensor comprises a non-image forming sensor configured to generate a signal indicative of the relative positioning of relatively low and high intensity portions of an optical signal incident on the sensor, in at least two dimensions;
the optical system is configured to direct diffuse reflections from a cornea of an eye under examination to an input face of the position-sensitive optical tracking sensor; and
the tracking controller is configured to utilize an intensity distribution signal from the position-sensitive optical tracking sensor to control an optical alignment function of the optical stage, relative to a cornea of an eye under examination.
